(19) United States
(12) Patent Application Publication
(10) Pub. No.: US 2018/0177625 A1
(43) Pub. Date: Jun. 28, 2018

(54) APPARATUS AND METHOD FOR TREATING MALE IMPOTENCE

(71) Applicant: Wei Zhang, Sunnyvale, CA (US)

(72) Inventor: Wei Zhang, Sunnyvale, CA (US)

(21) Appl. No.: 15/820,401

(22) Filed: Nov. 21, 2017

Related U.S. Application Data
(60) Provisional application No. 62/425,084, filed on Nov. 22, 2016.

(51) Int. Cl. A61F 5/41

(52) U.S. Cl. CPC A61F 5/41; A61F 2005/411

(57) ABSTRACT

The present invention strategically utilizes the sturdiness of thin wires enwrapped inside a thick layer of soft material such as silicone rubber which is smoother and softer than human tissue. Therefore, the device doesn't cause any damage or side effect and feels comfortable to user. The exposure of skin is 90% or higher, therefore solves the lack of sensation issue faced by prior art devices. The device is hard and yet flexible, the side walls or double bars are positioned toward ventral side of the device so that it is pleasing to the eye during use in addition to feeling good by the user.

### CROSS REFERENCE OF RELATED APPLICATIONS:

The present application claims the benefit of U.S. Provisional Application No. 62/425,084, filed on Nov. 22, 2016 entitled “An Improved Apparatus and Method for Treating Male Impotency”, which is hereby incorporated by reference in its entirety.

### STATEMENT REGARDING FEDERALLY SPONSORED RESEARCH OR DEVELOPMENT:

None.

### BACKGROUND OF THE INVENTIONS

This invention relates generally to a device and method curing male impotence, and more specifically, an improved non-invasive device curing male impotence with no or little side effect.

An estimated 25-30 million men in the United States are affected by impotency, or male erectile dysfunction (ED). In fact, on average about ten percent (10%) of the male population worldwide are affected by impotency. With more advanced age, such percentage increases significantly. For example, it is believed that for male population with an age of 40 years or older, the percentage of impotency is more than fifty percent (50%). For people with certain medical conditions, such as complications of diabetes, impotency is even more prevalent. Male impotency or male erectile dysfunction is usually not fatal. However, the emotional pain and suffering it brings to human kind should not be underestimated. Impotency could cause a person depression, anxiety, lack of self-confidence, sometimes anger, for some even thoughts of suicide. Therefore, an effective solution to treat impotency are in strong demand by both male and female populations, in order to restore male's self-esteem, assist woman who desire to conceive children, enhance couples' marriages, help family's overall physical and mental well beings, and for some, even save their lives.

Impotency is often caused by temporary mental state and psychological factors such as stress, fear, disinterest in sex, or certain temporary physical conditions caused by fatigue, lack of exercise, side effects of medication, etc. rather than permanent physical impairment. Therefore, most impotencies are curable regardless of the age of the person. In fact, 90% male with impotency issues can be cured and function normally.

Currently, there are various approaches of treating male impotency, some are intrusive (such as surgery), and others have side effects accompanying the usage of the treatment (such as medication). External mechanical devices are preferred by many because they offer more flexibility and are easier to test so the user can quickly determine the effectiveness of the treatment. However, existing external devices either are too expensive, difficult to use, or induce discomfort to the male user and/or his female partner. For example, external vacuum therapy is a noninvasive device, but one has to interrupt foreplay to put the pump cylinder onto the penis, secure the tension ring onto the penis, wait for the pump to create a vacuum in the cylinder to induce penis erection, remove the pump leaving the tension ring on the penis, then engage in sexual intercourse. It is inconvenient to both the male user and his female partner. There are other devices that help penile erections by providing stimulations to the penis. For example, Blakoe Ring is worn at the base of the penis, with its copper and zinc electrical conductors working with the damp skin of the penis to create impulses to help the erection. However, some users report that Blakoe Ring results in irritations to skin and thus is uncomfortable to wear. In addition, relying on putting an elastic ring around a penis to restrict penile blood flow to produce an erection has its limitations and disadvantages. If the restriction from the ring is not enough, it is ineffective in causing the erection. On the other hand, if the restriction from the ring is too much, it could cause numbness, cold, or even petechial to the user's penis. Moreover, none of these devices are effective in helping males with short or thin penises. On top of that, most existing devices are expensive, and require thorough cleaning in order to reuse the device.

In summary, existing devices and methods to cure male impotency either are invasive, have side effects, or are inconvenient, hard to use, uncomfortable, ineffective, or too expensive.

### BRIEF SUMMARY OF THE INVENTION

The present invention advantageously fills the aforementioned deficiencies by providing an effective, comfortable, easy to use, and low cost noninvasive device with no side effects to help people with impotence issues. This external device is easy to wear, comfortable to the skin, extremely low cost for consumers to use, and causes no side effects. In addition, it effectively helps those males who have small or thin penis. More importantly, compared to other similar apparatus for curing male impotence, the present invention increases the physical contact between male and female skins and thus enhances natural feelings in the users.

The present invention relates to an external device to cure male impotency. It has a soft exterior similar to a human skin so that it is not uncomfortable for the female partner. In some embodiments, the device only covers a small portion of the male penis so that the male's penis is largely exposed to the inner wall of the female partner's vagina during sexual intercourse to increase both the male user's and female partner's sexual arousal.

In one particular embodiment of the present invention, the device is made with silicone rubber or other flexible material, into a tube or sleeve shape (“Sleeve”) and placed on the outside of a penis. The length of the Sleeve can be either as long as the penis length or shorter than the penis for example at about ⅔ of the penis length. The Sleeve sits at or near the upper part of the scrotum and rear ⅔ of the penis shaft. With subcutaneous tissue very loose in this area, the silicone Sleeve can slide easily. An optional element is to include a string, rubber band, or other securing mechanism to loosely keep the Sleeve close to the Scrotum. The benefit of this design is several folds: i) Unlike Viagra, this device doesn't interfere with body's physiological function, and has no side effects—our experimental results on 28 patients indicate that after 5˜15 times of usage, the patients can naturally initiate and finish their sexual intercourse without any aid of the Sleeve; ii) The Sleeve is very convenient to use, economical, and effective to produce result instantly; and iii) The Sleeve doesn't cause any allergic reactions because refined silicone is very safe to human body. In fact some similar materials are even implemented inside human bodies, such as silicone implements or silicone injections, without any side effect.

In another embodiment of the present invention, the side of the Sleeve has one or more openings to expose more of the wearer's penis skin to physical contact and friction with Sleeve wearer's partner's skin. More importantly, such structure makes the putting device onto penis process significantly easier.

In still another embodiment of the present invention, one or more of the opening on the side of the Sleeve has one or more thin connecting material (“Connector”) to help reinforce the general shape of the Sleeve. Such Sleeve with Connectors provides improved sensation to Sleeve wearer during sexual intercourse while performing the Sleeve's function of providing certain restriction to the penis. Therefore, it produces improved results in curing impotency. At the same time, such structure maintains the easiness of putting the Sleeve onto penis.

In still another embodiment of the present invention, the sleeve is made of soft silicone material embedded within a thin wire or flexible stick to provide better support to the penis throughout the intercourse while the soft exterior ensures comfort to both male and female partners. Such silicone sheath with open windows exposes a majority portion of the penis to maintain maximum sensation of the user.

In still another embodiment of the present invention, the wire or stick wrapped with soft tissue like silicone or rubbers are shaped like an “S” bracket. It exposes even greater amount of the penis skin while providing sturdy support to the penis which in turn results even greater results in penis arousal and comfort to female partner.

It is therefore an object of the present invention to cure male impotency with an external device that is comfortable for the male user and his female partner, noninvasive, with little or no side effects. The advantage of the present invention is that it is convenient and effective immediately. Compared with sexual arousal pills widely sold on the market today, the device from the present invention is faster, safer, and offers longer term benefits by curing the male user's impotency eventually.

It is another object of the present invention to make an external device to cure male impotency that is low cost and convenient so that more male users can use them, discard them, and afford them readily.

It is still another object of this present invention to make an external device to cure male impotency while reducing the length of the device after the human male user's penis is elongated during sexual arousal to reduce any discomfort to the female partner.

It is still another object of this present invention to make an external device to cure male impotency with sleeve like elastic tube enwrapping a portion of the dysfunctional penis so that a substantial part of the front potion of the penis is still exposed and thus enables both the male and female partners enjoy intimacy during sexual intercourse.

It is still another object of this present invention to make an external device to cure male impotency with sleeve like elastic tube enwrapping the dysfunctional penis with one of more opening on the side of sleeve so that more of the penis skin is exposed to sleeve wearer's partner's skin and thus enables both the male and female partners enjoy greater skin friction and intimacy during sexual intercourse.

The present invention now will be described more fully hereinafter with reference to accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

The invention may take physical form in certain parts and arrangement of parts, a preferred embodiment of which will be described in detail in this specification and illustrated in the accompanying drawings which form a part hereof and wherein:

FIG. 1 is a photo of a silicone rubber Sheath with open windows.

FIG. 2 is a view in perspective of an equipment setup to make the silicone rubber Sleeve, Sheath, or S bracket and the corresponding process.

FIG. 3 includes photos of the silicone sheath with open windows shown at different angles.

FIG. 4 is a photo of an “S” shaped silicone bracket.

### DETAILED DESCRIPTION OF THE INVENTION

The present invention is directed to an external noninvasive device to cure male impotency at a low cost without side effects.

Referring now to the drawings wherein the showings are for purposes of illustrating a preferred embodiment of the invention only and not for purposes of limiting the same.

Several proto type of regular Sleeve made with silicone rubber were also produced and tested on patients. The sleeve has been cut into a shorter length so that it only covers approximately ⅔ of a penis, and thus exposes the front ⅓ of the penis which has most sensitivity, thus enabling greater sexual pleasure during the intercourse. One of the embodiments has a smaller end for the Sleeve while the other end is larger. In other embodiments, both ends of the Sleeve could have equal sizes in diameter, or both have different sizes in diameter. With the rapid advancement of material sciences, the elastic material for the Sleeve can be made much softer yet strong, elastic yet firm, comfortable, in various colors such as skin color. They could look and feel more and more like human skin such that it is difficult to tell that one is wearing such a device. The material can be made with disposable material so that user only uses once and discard afterwards. Alternatively, the device can also be made by material that can be re-used multiple times, provided that user disinfect it before usage such as boiling the Sleeve in hot water, or other type of disinfecting methods.

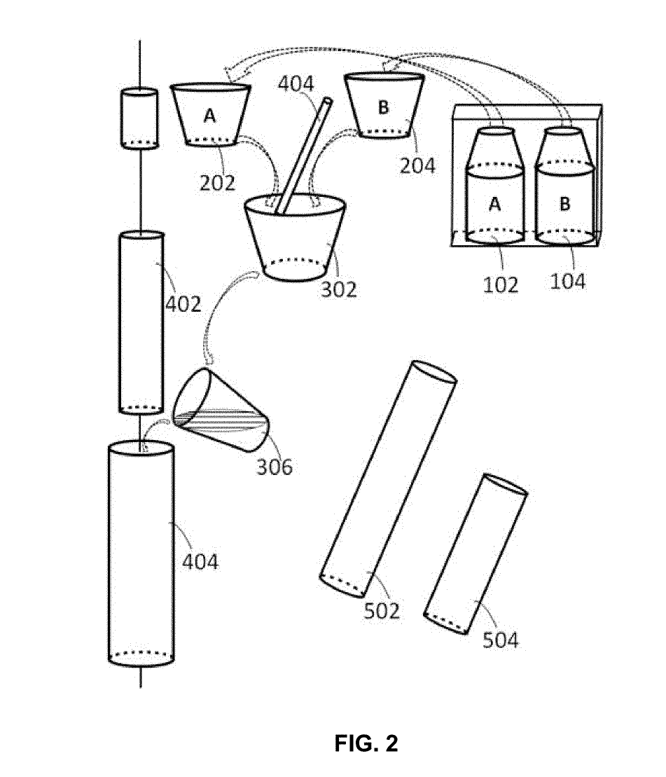

FIG. 2 illustrates the system and setup to manufacture a Sleeve using silicone rubber (here the commercially available silicone rubber material was purchased from Douglas & Sturges Co.). Liquid materials 102 and 104 in bottles A and B respectively is transferred into containers 202 and 204, respectively. The two liquids are mixed together in container 302, using a mixing rod 304. Then the mixed liquid 306 is poured into a cylindrical shaped mold container 404. A rod 402, with a lightly smaller diameter (for example, 5 mm smaller) than that of the mold container is inserted into the mold container to stir and mix the two liquids and then let it dry into a tube or sleeve shape. Another alternative is to simply shape the mold into a tube shape and pour the mixed silicone rubber liquid into it and let it dry. The resulting product is an elastic silicone rubber Sleeve either in full length as shown by 502, or a slightly shorter version, for example ⅔ of the penis shaft length as shown by 504. The length can also be adjusted by cutting the tube into a shorter length. The longer version helps prevent early semen leakage while the shorter version allows more intimacy and sensation in the penis glens area because it has more exposed penis glens area.

There are various ways to use the Sleeve; one of the usage embodiments is described in the following step: i) shave hair around the penis area and apply a lubricant on penis; ii) invert and fold the Sleeve, make it shorter. Choose a Sleeve with the same or similar dimension in its outer diameter as that of the penis in its relaxed state (the thickness of the Sleeve should be such that it is elastic and flexible yet strong, for example approximately 5 mm). Put middle and ring fingers of both hands in the hole of the Sleeve and dilate it, make the hole large enough to pass the penis, then unfold the Sleeve. Now the penis should erect and become hard and longer immediately. In addition, the user should feel very comfortable because the Sleeve is soft, smooth, feels almost like skin. The effect is quick and effective.

With the rapid advancement of modern technology, such Sleeves can be made more easily which can be adjusted and customized to individual user's specific size and preference in shape, length, width, elasticity, tightness, thickness and so on. For example, 3D printers can now produce soft rubber material in almost any shape one desires. Therefore, 3D printers could be the ideal equipment for producing customizable Sleeves.

Another embodiment is a silicone sheath or Sleeve in a color of human skin made using the equipment setup indicated in FIG. 2 using silicone gel material manufactured by Smooth-on. Its elasticity and softness is similar to that of a human organ, with smooth and fine texture. It is stretchable yet with strong pulling force. It is non-toxic and doesn't cause any allergic reaction, comfortable to wear, easy to sterilize by simply using boiling water. It is also low cost, appealing to the eye, and thus can be promoted easily and broadly. It can be made into any skin color to accommodate various race or individual preference. Some users tried it during an intercourse and their partner didn't realize the user was wearing the silicone sheath until was told afterwards.

However, simple silicone sheath (“Sleeve”) may have some drawbacks for certain users. For example, some users find the side of the Sleeve retards feeling of the penis during intercourse, and therefore reduce their pleasure of sexual contact with their partners. Others find it difficult to put the Sleeve onto penis and requires significant amount of lubricant.

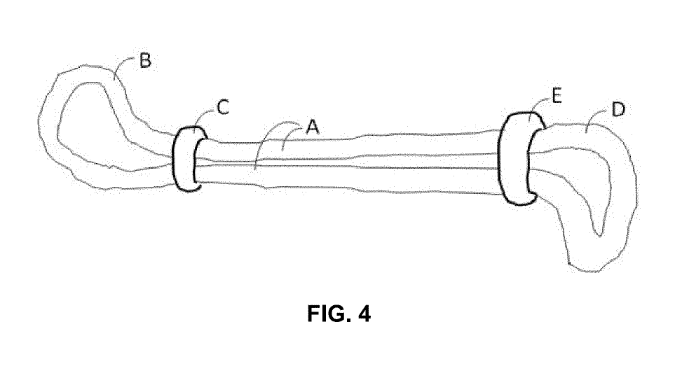

Our improved Sleeve has one or more openings on the side of the Sleeve so that more of the penis skin is exposed to the outside. For example, in a particular embodiment, the Sleeve has an opening both in its dorsal and ventral side separately. As a result, wearer is able to feel significantly improved pleasure through skin contact with that of wearer's partner. FIG. 4 is an embodiment of the equipment to mold a Sleeve with one or more openings on the side. Such high pressure applied to the side of the Sleeve during its curing step creates openings on the side of the Sleeve with smooth and soft edges. One can also use this method to create multiple openings arranged such that the remaining strips between various openings form one or more Connectors on the side of the Sleeve.

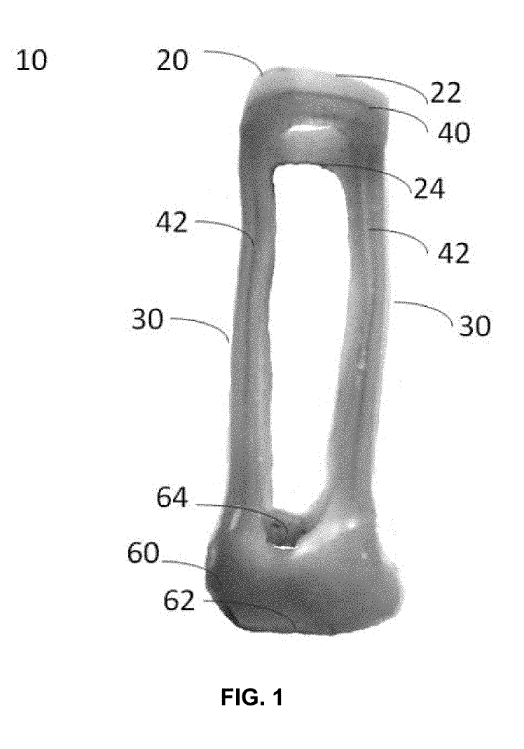

FIG. 1 is a picture of a silicone sheath with open windows. The sheath 10 has a soft exterior simulating human skin made of soft or elastic materials such as rubber, silicone rubber, etc. The sheath comprising a fore ring (near the tip of the penis) 20, a rear ring (near the root of the penis) 60, and at least a side wall 30. The side wall 30 may be shaped like a vertical strip of a cylinder having a smooth inside wall and outside wall concaving toward the center of the cylinder with edges tapering off to thinner thickness. This makes the side wall conform better to the penis shape. The fore ring 20 has a top portion (dorsal side) 22, a bottom portion (ventral side) 24, while the rear ring 60 has a top portion (dorsal side) 62, and a bottom portion (ventral side) 64. One or more stent segments are embedded inside the soft exterior to increase the rigidity of the sheath yet providing elasticity making it easy to put sheath onto penis. In one of the embodiments, stent is embedded on the side wall 30. There may be one or more side walls. In the embodiment shown in FIG. 1, two side walls 30 connect the fore ring and the rear ring. A stent 42 is embedded inside side wall 30 along a portion or the entire length of the side wall 30. The stent is made of a thin, somewhat stiff, yet flexible material so that it can hold the shape of the sheath yet allowing the sheath to strength to desired degree. The stent may be made of thin plastic rod, stainless steel wire or other rigid yet flexible material. To enable the side wall to stretch, the stent may be only extending through a portion of the side wall or comprising of several stent segments connected at joints that allow the overall side wall to change length. In one of the embodiments, the stent is made of stainless steel wire spirally coiled into tube like thin spring, connecting the fore ring and rear ring. When the sheath needs to be stretched, the stent spring stretches inside the soft surrounding side wall material. Meanwhile, the spring's elastic tension applies some force onto the fore ring and rear ring which helps arousal. The fore ring 20 may also embed a stent segment 40, and/or the rear ring 60 may embed a stent segment (not shown in FIG.1). Such segment(s) may comprise one or more pieces. For example, for the fore ring, stent 40 may form a near half circle inside the top portion 22. Alternatively, it may be made of a near-half circle inside the top portion 22 and a separate near-half circle inside the bottom portion 24. Other possibilities include a full circle or more than 2 segments together forming a near circle. Rear ring 60 could have some stent segments embedded in it or simply rely on the elasticity of the outside soft material to keep the sheath in place without any stent on the inside.

In one of the embodiments, the full length of the sheath may range 7 to 10 cm (which may stretch up to 18 cm when the penis is erected). In another embodiment, the full length may range 6 to 9 cm (which may stretch up to 17 cm when the penis is erected). Two side walls connect the fore ring and the rear ring. The two side walls are positioned closer toward the ventral (bottom) side to better support the penis and less visible to eye. We define the circumferential length of each ring from the mid-point of one side wall to the mid-point of another side wall. In one of the embodiments, the inner circumferential length of the top (dorsal) side of the rear ring is 5.2 cm. This portion may be made slightly thinner on the side closer to the side walls so user can easily enlarge this portion when initially putting the sheath onto penis. A more flexible rear ring also helps to eliminate or reduce the constrictive feelings during use. The inner circumferential length of the back (ventral) side of the real ring is 3.3 cm. This length is shorter than the front (dorsal) side because the two side walls are placed closer toward the back of the sheath. It is more pleasing to the eye and usage by hiding the silicone sheath from front view and placing the lateral side walls to the ventral side. The inner circumferential length of the front (dorsal) side of the fore ring is 4 cm, and the back (ventral) side of the fore ring is 2 cm, while the fore ring circumference is 6 cm. The front side of the fore ring is shorter than that of the rear ring to help catch the glens better and prevent it from slipping out. The width of the concave side wall is 0.8 cm, which helps to expose more of the skin to enhance the feeling. The outer circumference length of the front (dorsal) side of the rear ring is 7.5 cm (compared to 5.2 cm for the inner circumference length). The outer circumference length of the back (ventral) side of the rear ring is 3.8 cm (compared to 3.3 cm for the inner circumference length). The circumference length of the front (dorsal) side of the fore ring is 5.8 cm (compared to 4 cm for the inner circumference length) and that of the back (ventral) side of the fore ring is 3.2 cm (compared to 2 cm for the inner circumference length). In one of the embodiments, a stainless steel stent with a 1 mm diameter is embedded in the two side walls, back (ventral) side of the fore ring as well the rear ring. This way, the front (dorsal) side of the fore ring and rear ring are free from stents and thus can stretch wider. It enables easy attachment of the silicone sheath onto penis. The test results with participating patients were extremely good. For patients with severe impotence, they may still have issues using silicone sheath with open windows such that their glens still sway around without erection. An additional fixed or elastic band to attach the fore ring to the glens can solve this particular problem.

One drawback with the above improved Sleeve is that the opening becomes too big and thus unable to hold the Sleeve in its intended shape.

To reduce the impact of such drawbacks, we have designed a further improved Sleeve. We have created a Sleeve that either utilizes or leverages a brace which holds the Sleeve in proper place. Existing braces often use metal on the outside. We think metal is too hard and non-elastic, as well as hard to secure. In addition, it causes wearer's partner discomfort, pain, even injury. Therefore few such devices have been used. Our solution is to use a plastic wire with a little elasticity (either with or without soft material such as a layer of rubber coated on the outside of the wire), or other thin strip which is elastic yet with some rigidity. One embodiment is using thread to guide such plastic wire through the inside of the rubber material of the Sleeve. Therefore, the wire is embedded inside and surrounded by the rubber material of the Sleeve. This way, the Sleeve maintains a smooth inside and outside surface with fine texture. The direction of the wire or strip (collectively referred to as a “Connector”) is around the circumference of the tube shape of the Sleeve. Large scale manufacturing can use advanced techniques, such as molding the entire Sleeve with one or more Connectors along the circumference of the Sleeve. Such Connectors can be made the same material as the Sleeve and thus be molded together as the Sleeve. Alternatively, a Connector can be made of different material and attached via various methods to the Sleeve afterward the Sleeve is made. Other manufacturing methods can also be made available in the future, such as 3D printers using the same or different materials for the Sleeve sides and the Connectors. In one of the embodiments, a Sleeve has a thin Connector originating from the mid-point of a first side wall to the adjacent mid-point of the other side wall while another Connector originating from the opposite mid-point of the first side wall to the adjacent mid-point of the other side wall. These Connectors are in relaxed state but will stretch to longer length during erection. In some embodiments, there are two or more Connectors in parallel with each, creating multiple windows on the side. In other embodiments, the Connector may be made thicker than the side wall to prevent it from expanding too much. In still other embodiments, the Connector may be made of different material than the side walls.

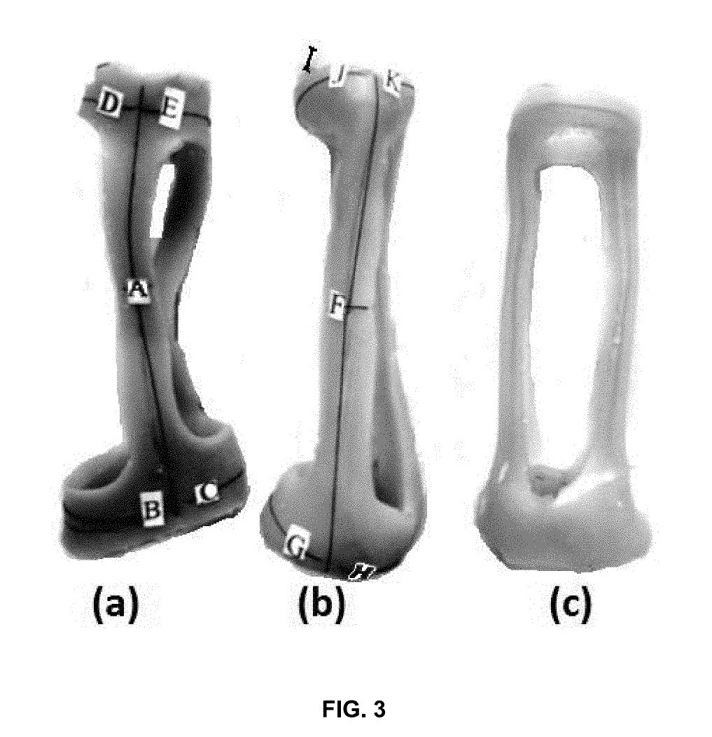

FIG. 3 dispicts pictures of a silicone rubber sheath with windows as well as a stainless steel wire embedded inside the silicone rubber, (a) its side view showing a side wall A, (b) another side view showing the other side wall F; (c) front view of the silicone rubber sheath with windows. DK is the front (dorsal) side of the fore ring, EJ is the back (ventral) side of the fore ring. BH is the front (dorsal) side of the rear ring, while CG is the back (ventral) side of the rear ring.

After properly attach the silicone rubber Sheath with windows device to a human male penis, the soft texture and the skin color of the Sleeve make it almost invisible and unnoticed via contact to wearer's partner. Lubrication is recommended when putting on the Sleeve. The thin plastic wire is barely visible to the naked eye.

FIG. 4 is another silicone rubber Sheath in the shape of an “S” letter, and thus is named as S-shaped silicone bracket. The purpose of this design is to maximize sensation by exposing more penis skins. This design is flexible and very easy to manufacture with a smooth overall structure and thus less likely to break. In one of the embodiments, we first connect the two ends of a stainless steel wire by soldering them together. Then bend the wire into a double bared s-shape with a half-ring at one end pointing up and another half-ring at the other end pointing down. The wire is has a diameter of 1.5 mm, is hard as a suturing needle so to better support the penis. The two side walls A extend from the rear end up to the front end bending up. This design is to avoid potential separation between the double side bars and the penis shaft so that it can hold the penis firmly. The outside portion of the wire is covered with a thick layer of silicone rubber coating with a diameter of 6 mm and in a texture that is soft like loose connective tissues of human body. Therefore, it is very comfortable for the user to wear. The s-shaped bracket starts with a half ring and ends with a half ring, with two bars A connecting them forming a sturdy shaft. The front half ring B bends upward and is located at the front (dorsal) side of the bracket to hold in the coronal sulcus. The front half ring is held together by a small latch ring C. The latch ring serves to hold the glands and keep it flat on the shaft. The rear half ring D bends down and is located on the down (ventral) side of the bracket, with a small latch ring E keeping the shaft further integrated together. The small latch rings can slide to and from between the two end rings so each ring can be adjusted to accommodate different size of penis. Thus, the s-shaped silicone bracket works well with various penis size and type. Thus, it is very convenient for a variety of users without the need of using lubrication. As the thickness of the double bar and two half-rings are only 6 mm in diameter, it enables the exposure of penis skin to as much as 90% or more. Thus, user can feel most part located in the back (ventral) side of penis when wearing the s-shaped silicone bracket. In addition, it also has a good concealment and privacy effect since the double bars are positioned toward the back (ventral) side. Therefore, it is very popular among subject who tried the silicone bracket. In order to enable the s-shaped silicone bracket stretch as needed, one may use stretchable yet rigid and flexible wire instead. One of the embodiments is using a thin spiral wire spring which is stretchable yet applies tension when stretched so to induce additional arousal.

In summary, we have made prototypes of various embodiments of the design, including but not limited to Brackets, Braces and Sleeves. One of the embodiments is a brace which hold penis in place. Another prototype is a simple skin colored sheath. Still another prototype is a Sleeve with one window or opening on the side. Still another prototype is a Sleeve with one window on the side and a thin connector holding the slide of Sleeve in place. The opening is almost the entire length of the Sleeve. With such a large opening, the Sleeve behaves essentially like a brace. Another group of prototypes includes two double windowed Sleeves. These two Sleeves have basically built-in Connector that hold the side of the Sleeve together. Another group of prototype is a darker colored Sleeve with a built-in Connector. Another embodiment is Sleeve with open window. Still another embodiment is s-shaped silicone rubber bracket.

We'll explain again how to properly put a brace onto penis and where to position the fore ring portion and real ring portion. A brace has a head portion, neck portion, body portion, and tail portion. The proper procedure is first putting on the rear ring or half ring, with the next step as putting the fore (or half) ring or head of the brace right onto the penis head. The bottom (ventral) side of the fore ring should sit along the peripheral of the penis head.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

We claim:

1. An external device to cure male impotency, comprising: an elastic tube with a first opening; a second opening; and a side wall with an outer diameter; and an inner diameter, wherein the inner diameter is slightly smaller than the outer diameter.

2. An external device to cure male impotency, as described in claim 1, further comprising: one or more side openings in the side wall of the tube.

3. An external device to cure male impotency, as described in claim 2, further comprising: at least one string made of the same or different material as the tube with one end connected to an edge of a side opening and another end connected to another edge of the same side opening.

5. A device as in claim 2, wherein the side opening is made such that the remaining portion on the side wall comprises one or more vertical strips connecting the first opening and the second opening of the tube.

6. A device as in claim 5, further comprising, one or more segments of thin wire are embedded inside the side wall of the tube.

7. A device as in claim 6, wherein the wire is stainless steel.

8. A device in claim 6 wherein the elastic material is silicone rubber.

9. A device as in claim 6, further comprising, at least a full or partial ring embedded at the first opening or the second opening.

10. A device as in claim 9, wherein there are two strips left on the side wall where the two strips are positioned closer to each other on one side than the other side.

11. A device as in claim 10, wherein the first opening and the second opening have different size.

12. A device as in claim 6, wherein the wire comprises a spring-like structure.

13. An external device to cure male impotency, comprising: a thin sturdy wire, a soft coating covering the wire, the wire shaped like an “S” with double bars extending from a first loop formed on a first end to a second loop on a second end.

14. A device as in claim 13, wherein the soft coating is silicone rubber.

15. A device as in claim 13, wherein the wire is segmented and connected through one or more connectors.

16. A device as in claim 13, wherein at least a portion of the wire comprises a thin spring structure.

17. A device as in claim 13, wherein the first loop bends in a first direction away from the plane of the double bar and the second loop bends toward the opposite direction.

18. A device as in claim 17, further comprising at least a latch near either the first loop or the second loop.

19. A device as in claim 18, further comprising a second latch near the other loop.
